(19) United States
(12) Patent Application Publication
(10) Pub. No.: US 2022/0152135 A1
(43) Pub. Date: May 19, 2022

(54) COMPOSITIONS COMPRISING ALGAE POWDER AND USES THEREOF

(71) Applicant: ALGATECHNOLOGIES LTD., Kibbutz Ketura (IL)

(72) Inventor: Yael CARRASSO, Kibbutz Yotvata (IL)

(73) Assignee: ALGATECHNOLOGIES LTD., Kibbutz Ketura (IL)

(21) Appl. No.: 17/435,763

(22) PCT Filed: Mar. 3, 2020
(86) PCT No.: PCT/IL2020/050241

§ 371 Date: Sep. 2, 2021

Related U.S. Application Data
(60) Provisional application No. 62/813,056, filed on Mar. 3, 2019.
(60) Provisional application No. 62/841,860, filed on May 2, 2019.

(51) Int. Cl. A61K 36/05; A61K 33/06; A61K 9/28; A61K 31/4415; A61K 31/122

(52) U.S. Cl. CPC A61K 36/05; A61K 33/06; A61K 9/2866; A61K 31/122; A61K 9/282; A61K 31/4415; A61K 9/2813; A61K 9/286

(57) ABSTRACT

The present invention is directed to a composition made of a microalgae powder, and a filler or a filler and magnesium. The composition may further include a wax material, an emulsifier, an antioxidant, or their combinations.

### CROSS-REFERENCE TO RELATED APPLICATIONS

This application claims the benefit of priority of U.S. Provisional Patent Application No. 62/813,056 titled “COMPOSITIONS COMPRISING ALGAE POWDER AND USES THEREOF”, filed Mar. 3, 2019, and of U.S. Provisional Patent Application No. 62/841,860 titled “COMPOSITION OF MAGNESIUM AND ASTAXANTHIN”, filed May 2, 2019, the contents of which are incorporated herein by reference in their entirety.

### FIELD OF INVENTION

The present invention is in the field of microalgae biotechnology.

### BACKGROUND

Microalgae grow in either marine or freshwater systems. They are unicellular species which exist individually, or in chains or groups. Microalgae are capable of performing photosynthesis, and are primary producers in the oceans that convert water and carbon dioxide to biomass and oxygen. Microalgae species produce unique products such as carotenoids, antioxidants, fatty acids, enzymes, polymers, peptide, and sterols. Astaxanthin, as a non-limiting example, is a carotenoid found in microalgae and has been shown to be beneficial in the prevention and treatment of various diseases such as cancers, chronic inflammatory diseases, metabolic syndrome, diabetes, eye diseases, skin diseases, among others.

Haematococcus pluvialis is a freshwater species of Chlorophyta from the family Haematococcaceae containing common carotenoids, fatty acids, proteins, carbohydrates, and minerals. This species is well known for its high content of the strong antioxidant astaxanthin, which is important in aquaculture, and cosmetics.

Magnesium and astaxanthin are two dietary supplements that are beneficial to people engaged in sports. Preventing cramping and shortening recovery times after exertion, are just two examples for the beneficial effects attributed to these compounds. Therefore, the incentive to formulate both in a single dietary supplement is clear. Microalgae extracts or powders are known to be rich on astaxanthin as well as on other important dietary ingredients, e.g., pigments, anti-oxidants, unsaturated fatty acids, etc. However, the health beneficiary attributes of such microalgal powders, including the strong antioxidant activity of astaxanthin, are severely deteriorated when formulated with magnesium salts due to the latter's tendency to oxidize. Therefore, there is still a biotechnological challenge so as to formulate a combination of a microalgal powder or extract having high levels of reduced/non-oxidized astaxanthin and magnesium salts.

### SUMMARY

The present invention, in some embodiments thereof, is directed to a composition comprising: a microalgae powder, a filler, an excipient, and any one of a wax material, an emulsifier, an antioxidant, or a combination thereof.

The present invention, in some embodiments thereof, is directed to a composition comprising astaxanthin and magnesium. In some embodiments, the astaxanthin is derived from a microalgae lysate. In some embodiments, the composition further comprises a microalgae powder.

The invention, in some embodiments thereof, is based, in part, on the surprising finding that a specific ratio between the whole microalgae powder and: a filler or a filler and magnesium rendered it substantially more stable and processable (e.g., pelletable, feasible under large-scale production, etc.).

The invention is based, in part, on the surprising finding that although microalgae-derived astaxanthin is known to have great tendency to oxidize in the presence of magnesium, it maintained stability levels of more than 90% (i.e., low rate of oxidation) for a period of months when the magnesium was ionically bound to an oxyanion.

According to a first aspect, there is provided a composition comprising: (a) a microalgae powder in an amount of 5-30%, by weight; and (b) a filler or (c) a filler and magnesium, wherein (b) or (c) is in the amount of 60-85%, by weight.

According to another aspect, there is provided a process for preparing a composition, the process comprising mixing: (a) a microalgae powder in an amount of 5-30%, by weight; and (b) a filler or (c) a filler and magnesium in an amount of 60-85%, by weight, thereby preparing the composition.

In some embodiments, the composition is in the form of a tablet.

In some embodiments, the composition has increased stability for a period of at least 6 months.

In some embodiments, the composition further comprises: an excipient, a wax material, an emulsifier, an antioxidant, or a combination thereof.

In some embodiments, the microalgae powder comprises at least one carotenoid.

In some embodiments, the at least one carotenoid is selected from the group consisting of: astaxanthin, lutein, and fucoxanthin.

In some embodiments, astaxanthin is present in the composition in an amount of 1-8%, by weight.

In some embodiments, the filler is selected from the group consisting of: inulin, maltodextrin, gum acacia, and any combination thereof.

In some embodiments, the filler comprises inulin, maltodextrin, and gum acacia.

In some embodiments, the filler is inulin.

In some embodiments, inulin is present in the composition in an amount of 35-75%, by weight.

In some embodiments, the filler comprises a dietary fiber.

In some embodiments, the wax material is selected from the group consisting of: carnauba wax, beeswax, candelilla wax, rice wax, lanolin wax, and any combination thereof.

In some embodiments, the wax material is carnauba wax.

In some embodiments, carnauba wax is present in the composition in an amount of 1-10%, by weight.

In some embodiments, the emulsifier is hydroxypropyl cellulose (HPC), sodium croscarmellose, rice extract, or any combination thereof.

In some embodiments, the emulsifier is present in the composition in an amount of 1-12%, by weight.

In some embodiments, the antioxidant is selected from the group consisting of: sodium ascorbate, carnosic acid, rosemary extract, tocopherol, and any combination thereof.

In some embodiments, the antioxidant is present in the composition in an amount of 0.01-2%, by weight.

In some embodiments, the excipient is silicon dioxide, starch, or a combination thereof.

In some embodiments, the excipient is present in the composition in an amount of the amount 1-12%, by weight.

In some embodiments, the dietary fiber and the wax material are present in the composition in a ratio ranging from 80:1 (w/w) to 2:1 (w/w).

In some embodiments, the microalgae powder is present in the composition in an amount of 8-24%, by weight.

In some embodiments, the magnesium is ionically bound to an oxyanion.

In some embodiments, the magnesium is magnesium glycerophosphate.

In some embodiments, magnesium is present in the composition in an amount of 1-50%, by weight.

In some embodiments, the microalgae is Haematococcus pluvialis.

In some embodiments, the composition further comprises an amino acid in an amount of 0.5-2%, by weight.

In some embodiments, the amino acid is selected from the group consisting of: Leucine and Valine.

In some embodiments, the composition further comprises a coat or a shell, wherein the coat or shell is present in the composition in an amount of 0.1-8%, by dry weight.

In some embodiments, the composition further comprises one or more additives.

In some embodiments, the one or more additives are selected from the group consisting of: a coloring agent, a flavoring agent, a vitamin, and a mineral.

In some embodiments, the coloring agent is present in the composition in an amount of 0.01-5%, by weight.

In some embodiments, the coloring agent comprises an anthocyanin.

In some embodiments, the flavoring agent is present in the composition in an amount of 0.5-2.5%, by weight.

In some embodiments, the composition is formulated for oral administration.

In some embodiments, the process further comprises the of mixing the composition with: an excipient, a wax material, an emulsifier, an antioxidant, or any combination thereof.

In some embodiments, the prepared composition is the herein disclosed composition.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### DETAILED DESCRIPTION

The present invention, in some embodiments thereof, is directed to a composition comprising: a microalgae powder, and: a filler or a filler and magnesium. In some embodiments, the composition further comprises a wax material, an emulsifier, an antioxidant, or a combination thereof. In some embodiments, the composition further comprises an excipient.

Microalgae

In some embodiments, the composition comprises a dried and grinned microalgae lysate, or a fraction thereof, in a powder form (‘microalgae powder’).

As used herein, the term “microalgae” refers to any unicellular, photosynthetic microorganism. In one embodiment, the microalgae are wild type microalgae. In another embodiment, the microalgae are genetically modified microalgae. In some embodiments, the microalgae are of the genus Haematococcus. In some embodiments, the microalgae are of the species H. pluvialis. Other, non-limiting examples of microalgae powders of which are also applicable according to the present invention include, but are not limited to, Phaeodactylum tricornutum, Navicula pelliculosa, Amphora, Isochrysis aff. Galbana, Odontella aurita, Nitzscia closterium, Cyhndrotheca closterium, Chaetoseros sp., and Emiliania huxleyi and any combination thereof.

In some embodiments, the microalgae powder comprises astaxanthin. In some embodiments, the microalgae powder comprises astaxanthin in an amount of 1-5% by dry weight, 1.2-5.8% by weight, 1.5-6.2% by dry weight, 2-4.5% by dry weight, 2.4-5.5% by dry weight, 2.9-4.5% by dry weight, 3-5.7% by dry weight, 3.5-6.2% by dry weight, 4-7.4% by dry weight, or 4.3-8% by dry weight of the microalgae powder. Each possibility represents a separate embodiment of the invention. In some embodiments, the microalgae powder comprises astaxanthin in an amount of at least 1% by weight, at least 2% by weight, at least 3% by weight, at least 4.5% by weight, at least 5% by weight, at least 6.5% by weight, at least 7% by weight, at least 7.5% by weight, or at least 8% by weight, and any value and range therebetween. Each possibility represents a separate embodiment of the invention.

In some embodiments, the disclosed composition comprises a microalgae powder in an amount of 0.1-30%. In some embodiments, the disclosed composition comprises a microalgae powder in an amount of 1-28% by weight, 2-19% by weight,3-20% by weight, 8-28% by weight, 2.9-32% by weight, 5-24% by weight, 6-25% by weight, 2-25% by weight, or 7-29% by weight. In some embodiments, the composition comprises a microalgae powder in an amount of at least 0.1% by weight, at least 0.5% by weight, at least 1% by weight, at least 2% by weight, at least 4% by weight, at least 7% by weight, at least 9% by weight, at least 12% by weight, at least 15% by weight, at least 20% by weight, at least 23% by weight, at least 25% by weight, at least 27% by weight, at least 29% by weight, or at least 30% by weight, and any range and value therebetween. Each possibility represents a separate embodiment of the invention.

In one embodiment, the phrase “by weight” includes by weight of the total composition, by weight of the dry composition, or by weight of the microalgae powder. In one embodiment, the phrase “a dry composition” comprises less than 1% water, less than 0.1% water, or less than 0.05% water, by weight, and any value or range therebetween. Each possibility represents a separate embodiment of the invention. In some embodiments, a dry composition comprises, 0.5-5% water, 0.7-4.5% water, 0.6-3.5% water, 1-4.5% water by weight. Each possibility represents a separate embodiment of the invention.

As used herein, the “microalgae powder” encompasses any powder obtained or derived from microalgae, selected from: whole microalgae, whole microalgae lysate, whole microalgae extract, fractions thereof, materials extracted therefrom, and any combination thereof. In one embodiment, microalgae can be harvested prior to subsequent processing, such as but not limited to lysis, milling, and extraction, by any conventional means including, but not limited to mechanical lysis, filtration, air flotation and centrifugation.

In one embodiment, the microalgae powder comprises a microalgae biomass. In some embodiments, the biomass is a dried biomass.

Methods of large scale microalgae cultivation, such as Haematococcus cells are known in the art, for instance, in U.S. Pat. No. 6,022,701, which is incorporated herein by reference in its entirety.

Microalgae lysis (e.g., for obtaining oleoresin) may be carried out by any means known in the art. In one embodiment, the lysis is a mechanical process. In another embodiment, the lysis is carried out in the presence of an organic solvent. In one embodiment, the organic solvent is at least partially miscible in water. In another embodiment, the organic solvent is immiscible in water. Organic solvents are common and would be apparent to one of ordinary skill in the art, non-limiting example of which, include, but is not limited to ethanol, and ethyl acetate.

As defined herein, the term “fraction” refers to a result of any process by which multiple substances of a mixture, such as a whole cell lysate, are divided and subsequently collected into a number of smaller quantities (i.e., fractions) each comprised of several components sharing one or more specific properties. Methods of fractionation are common and would be apparent to one skilled in the art, non-limiting examples of which include adsorption, capillary electrophoresis, centrifugation, cyclonic separation, chromatography, crystallization, decantation, demister, distillation, drying, electrophoresis, electrostatic separation, elutriation, evaporation, extraction, field flow fractionation, flotation, flocculation, filtration, fractional filtration, fractional freezing, oil-water separation, magnetic separation, precipitation, recrystallization, scrubbing, sedimentation, sieving, stripping, sublimation, vapor-liquid separation, winnowing and zone refining, and others.

Filler

In some embodiments, the composition comprises a filler. The terms “filler”, “bulking agent”, and “diluent” as used herein are interchangeable, and refer to non-active ingredient in a composition, such as but not limited to a nutraceutical or pharmaceutical composition, which renders either long-term stabilization or therapeutic enhancement of the active ingredient in the final dosage form, enables or improves absorption, increases solubility, reduces viscosity, or any combination thereof.

In some embodiments, the filler comprises a dietary fiber. In some embodiments, the filler comprises a polysaccharide. In some embodiments, the filler comprises a dietary fiber, a polysaccharide, or both. As used herein, the term “dietary fiber” refers to non-starch carbohydrates found in plants or other organisms containing cell walls. In some embodiment, a dietary fiber is water soluble. In some embodiments, a dietary fiber is water insoluble, e.g., not able to be dissolved in water. In some embodiments, a dietary fiber further comprises a glycopeptide.

In some embodiments, the composition comprises a single filler. In some embodiments, the composition comprises at least 2 fillers. As used herein, at least 2 comprises 2-3, 3-5, or 3-7 types of filler, and any value and range therebetween. In some embodiments, the composition comprises a filler selected from: inulin, maltodextrin, gum acacia, and any combination thereof. In some embodiments, the composition comprises inulin, maltodextrin, and gum acacia. In some embodiments, the composition comprises inulin.

As used herein, “inulin” refers to a group of naturally occurring fructans (e.g., fructan-comprising polysaccharides) belonging to a class of dietary fibers produced by many types of plants.

In some embodiments, the disclosed composition comprises a filler, such as a dietary fiber, for example inulin, in an amount of at least 35%, at least 40%, at least 50%, at least 60%, at least 65%, at least 70%, at least 75%, or at least 80%, by weight, and any value and range therebetween. Each possibility represents a separate embodiment of the invention. In some embodiments, the composition comprises a filler, such as a dietary fiber in an amount of 30-80%, 35-75%, 40-70%, 55-75%, 45-65%, 60-80%, or 65-85% by weight. Each possibility represents a separate embodiment of the invention.

In some embodiment, the composition comprises a mixture of dietary fibers, polysaccharides, or any combination thereof, in an amount of at least 35%, at least 40%, at least 45%, at least 50%, at least 60%, at least 65%, at least 70%, at least 75%, or at least 80%, by weight, and any value and range therebetween. In some embodiment, the composition comprises a mixture of dietary fibers, polysaccharides, or any combination thereof, in an amount of 50-70%, 45-75%, 60-85%, or 55-85% by weight. Each possibility represents a separate embodiment of the invention. As a non-limiting example, the composition comprises a mixture comprising inulin (i.e., dietary fiber) in an amount of 40% by weight, maltodextrin (i.e., polysaccharide) in an amount of 25% by weight, and gum acacia (i.e., a mixture of polysaccharides and glycoproteins).

In some embodiments, the composition comprises inulin in an amount of 42-75% by weight, 43-76% by weight, 45-77% by weight, 55-78% by weight, 54-79% by weight, 60-80% by weight, 57-72% by weight, 59-85% by weight, 60-76% by weight, or 61-77% by weight. Each possibility represents a separate embodiment of the invention. In some embodiments, the composition comprises inulin in an amount of at least 35% by weight, at least 40% by weight, at least 45% by weight, at least 50% by weight, at least 55% by weight, at least 60% by weight, at least 65% by weight, at least 70% by weight, at least 75% by weight, at least 80% by weight, or at least 85% by weight, and any value and range therebetween. Each possibility represents a separate embodiment of the invention.

Wax Material

As used herein, the term “wax material” encompasses any one of organic compounds which are characterized as lipophilic, ductile solids in ambient temperatures. In some embodiments, any wax material known in the art can be used as the wax material in the composition of the invention. In some embodiments, the wax material is selected from animal, vegetable, mineral and synthetic waxes. In some embodiments, the wax material is selected from carnauba wax, beeswax, candelilla wax, rice wax, lanolin wax, and any combination thereof.

In some embodiments, the composition comprises carnauba wax. In some embodiments, the wax material is carnauba wax.

In some embodiments, the composition comprises a wax material in an amount of at least 1% by weight, at least 2% by weight, at least 3% by weight, at least 4% by weight, at least 5% by weight, at least 6% by weight, at least 7% by weight, at least 8% by weight, at least 9% by weight, at least 10% by weight, at least 11% by weight, at least 12% by weight, at least 13% by weight, at least 14% by weight, at least 15% by weight, at least 16% by weight, at least 17% by weight, at least 18% by weight, at least 19% by weight, or at least 20% by weight, and any value or range therebetween. Each possibility represents a separate embodiment of the invention. In some embodiments, the composition comprises a wax material in an amount of 1-25% by weight, 2-20% by weight, 3-18% by weight, 4-16% by weight, 5-15% by weight, 6-20% by weight, or 7-19% by weight. Each possibility represents a separate embodiment of the invention.

In some embodiments, the composition comprises carnauba wax in an amount of at least 1% by weight, at least 3% by weight, at least 5% by weight, at least 7% by weight, at least 9% by weight, at least 11% by weight, at least 13% by weight, at least 15% by weight, at least 17%, at least 19%, or at least 20%, and any value and range therebetween. Each possibility represents a separate embodiment of the invention. In some embodiments, the composition comprises carnauba wax in an amount of 1-15% by weight, 2-18% by weight, 3-17% by weight, 4-19% by weight, 5-20% by weight, 6-19% by weight, 7-15% by weight, 8-16% by weight, 9-21% by weight, 12-22% by weight, 10-18% by weight, or 1-20% by weight. Each possibility represents a separate embodiment of the invention.

In some embodiments, the present invention is directed to a composition comprising a filler, such as a dietary fiber or a mixture thereof, and a wax material or a mixture thereof. In some embodiments, the filler comprises a dietary fiber. In some embodiments, the filler comprises a mixture of dietary fibers. In some embodiments, the filler, such as a dietary fiber or a mixture thereof, and the wax material or a mixture thereof are present in the composition in ratio of at least 80:1 (w/w), at least 75:1 (w/w), at least 70:1 (w/w), at least 65:1 (w/w), at least 60:1 (w/w), at least 55:1 (w/w), at least 50:1 (w/w), at least 45:1 (w/w), at least 40:1 (w/w), at least 35:1 (w/w), at least 30:1 (w/w), at least 25:1 (w/w), at least 20:1 (w/w), at least 15:1 (w/w), at least 10:1 (w/w), at least 5:1 (w/w), at least 4:1(w/w), at least 3:1 (w/w), or at least 2:1 (w/w), and any value and range therebetween. Each possibility represents a separate embodiment of the invention. In some embodiments, the filler, such as a dietary fiber or a mixture thereof, and the wax material or a mixture thereof are present in the composition in a ratio ranging from 80:1 (w/w) to 2:1 (w/w), 75:1 (w/w) to 3:1 (w/w), 70:1 (w/w) to 5:1 (w/w), 65:1 (w/w) to 10:1 (w/w), 60:1 (w/w) to 10:1 (w/w), 55:1 (w/w) to 2:1 (w/w), 50:1 (w/w) to 3:1 (w/w), 45:1 (w/w) to 2:1 (w/w), 40:1 (w/w) to 3:1 (w/w), 35:1 (w/w) to 10:1 (w/w), 30:1 (w/w) to 2:1 (w/w), 25:1 (w/w) to 3:1 (w/w), 20:1 (w/w) to 2:1 (w/w), 15:1 (w/w) to 3:1 (w/w), 11:1 (w/w) to 2:1 (w/w), or 5:1 (w/w) to 2:1 (w/w). Each possibility represents a separate embodiment of the invention.

Excipient

In some embodiment, the composition comprises an excipient. As used herein, the term “excipient” refers to any component of a composition that is not the active agent.

The terms “carrier”, “excipient”, or “adjuvant” are interchangeable.

As used herein, the term “acceptable excipient” refers to non-toxic, inert solid, semi-solid liquid filler, diluent, encapsulating material, formulation auxiliary of any type, or simply a sterile aqueous medium, such as saline. Some examples of the materials that can serve as acceptable excipient are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as palm oil (e.g. non-hydrogenated palm oil), peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, and agar.

In some embodiments, the composition comprises silicon dioxide. In some embodiments, the excipient is silicon dioxide.

In some embodiments, the composition comprises an excipient, for example silicon dioxide, in an amount of 0.5-4.5% by weight, 0.5-4% by weight, 0.5-3.5% by weight, 1-4.5% by weight, 1-4% by weight, 1.5-4.5% by weight, 2-4.5% by weight, 2-5% by weight, or 2-5.5% by weight. In some embodiments, the composition comprises an excipient, for example silicon dioxide, in an amount of at least 0.5% by weight, at least 1% by weight, at least 1.5% by weight, at least 2% by weight, at least 3% by weight, at least 4% by weight, at least 4.5% by weight, or at least 5% by weight, and any value and range therebetween. Each possibility represents a separate embodiment of the invention.

Emulsifier

In some embodiments, the composition comprises one or more emulsifiers. As used herein, the term “emulsifier” encompasses any substance which has the ability to stabilize an emulsion. In some embodiments, the emulsifier is a surface active agent. In some embodiments, the emulsifier is a surfactant.

As used herein, one or more emulsifiers comprises at least 2, at least 3, or at least 4 emulsifiers, and any range and value therebetween. In some embodiments, one or more emulsifiers comprises 1-3, 2-4, or 3-5 emulsifiers. Each possibility represents a separate embodiment of the invention.

In some embodiments, the emulsifier is selected from: HPC, sodium croscarmellose, rice extract (e.g., Nu rice) and any combination thereof. In some embodiments, the composition comprises HPC. In some embodiments, the composition comprises sodium croscarmellose. In some embodiments, the composition comprises HPC and sodium croscarmellose.

In some embodiments, the composition comprises HPC in an amount of at least 1% by weight, at least 2% by weight, at least 3% by weight, at least 4% by weight, at least 6% by weight, or at least 8% by weight, and any range therebetween. In some embodiments, the composition comprises HPC in an amount of 1-5% by weight, 2-6% by weight, or 3-8% by weight. Each possibility represents a separate embodiment of the invention.

In some embodiments, the composition comprises sodium croscarmellose in an amount of at least 0.5% by weight, at least 1% by weight, at least 1.5% by weight, at least 2% by weight, at least 2.5% by weight, at least 3% by weight, at least 3.5% by weight, at least 4% by weight or at least 4.5% by weight, and any range therebetween. In some embodiments, the composition comprises sodium croscarmellose in an amount of 0.5-2.5% by weight, 1-3% by weight, or 1.5-4.5% by weight. Each possibility represents a separate embodiment of the invention.

In some embodiments, the composition comprises HPC and sodium croscarmellose in a cumulative amount of at least 2% by weight, at least 3% by weight, at least 4% by weight, at least 5% by weight, at least 7% by weight, at least 9% by weight, at least 10% by weight, at least 6% by weight, at least 11% by weight, at least 12% by weight, at least 13% by weight, at least 14% by weight, or at least 15% by weight, and any range therebetween. In some embodiments, the composition comprises HPC and sodium croscarmellose in a cumulative amount of 2-8% by weight, 3-10% by weight, 4-11%, or 5-15% by weight. Each possibility represents a separate embodiment of the invention.

In some embodiments, HPC and sodium croscarmellose are present in the disclosed composition in weight per weight ratio, ranging from 4:1 to 1:1. In some embodiments, the ratio of HPC to sodium croscarmellose ranges from 4:1 to 2:1, 4:1 to 4:3, 4:1 to 4:2.5, or 4:1 to 4:4. Each possibility represents a separate embodiment of the invention.

In some embodiments, the composition comprises starch. In some embodiments, starch is present in the herein disclosed composition as a filler, an emulsifier, or both. In some embodiments, the composition comprises starch in an amount ranging from 1 to 12%, by weight.

Antioxidant

In some embodiments, the composition comprises an antioxidant. As used herein, the term “antioxidant” refers to any compound capable of inhibiting, decreasing, reducing, slowing, or preventing oxidation. In some embodiments, the antioxidant comprises a mineral salt of ascorbic acid. In some embodiments, the antioxidant comprises sodium ascorbate. In some embodiments, the antioxidant is sodium ascorbate. In some embodiments, the antioxidant is carnosic acid. In some embodiments, the antioxidant is a rosemary extract. In some embodiments, the antioxidant is a tocopherol, or a mixture thereof. In some embodiments, the antioxidant comprises: a mineral salt of ascorbic acid, sodium ascorbate, carnosic acid, a rosemary extract, a tocopherol, and any combination thereof.

In some embodiments, the composition comprises sodium ascorbate. In some embodiments, the composition comprises sodium ascorbate in an amount of at least 0.5% by weight, at least 1% by weight, at least 1.5% by weight, at least 2% by weight, at least 2.5% by weight, at least 3% by weight, or at least 5% by weight, and any range therebetween. In some embodiments, the composition comprises sodium ascorbate in an amount of 0.5-1.5% by weight, 1-3% by weight, or 1.5-5% by weight. Each possibility represents a separate embodiment of the invention.

Magnesium

According to some embodiments, the composition comprises magnesium. In some embodiments, the magnesium cation is ionically bound to an anion comprising an oxygen ion.

In some embodiments, magnesium is bound to a divalent anion.

In some embodiments, divalent anion comprises two or more oxygen ions. In some embodiments, magnesium is bound to a divalent anion comprising two or more oxygen ions.

In some embodiments, the divalent anion comprises a divalent oxyanion. In some embodiments, magnesium is bound to a divalent anion comprising a divalent oxyanion. In some embodiments, the oxyanion is selected from the group consisting of: phosphate, carbonate, and sulphate, borate, nitrate, chromate, arsenate, and ferrate.

In some embodiments, the anion is covalently linked to a carbon chain. As used herein, a carbon chain comprises two or more carbon atoms. In one embodiment, the carbon chain is an alcohol. In one embodiment, the carbon chain is a lipid. In one embodiment, the carbon chain is a fatty acid. In one embodiment, the carbon chain is an amino acid. In one embodiment, the carbon chain is a polymer.

In some embodiments, the anion is glycerophosphate. In some embodiments, the oxyanion is glycerophosphate. In some embodiments, magnesium is ionically bound to glycerophosphate. In some embodiments, magnesium salt of the invention comprises magnesium glycerophosphate.

According to another embodiment, the anion is selected form: pidolate, citrate, stearate, aspartate, gluconate, glycinate, lactate, levulinate, malate, and orotate.

In some embodiments, the composition comprises magnesium at an amount of 0.5-10% by dry weight, 7-15% by dry weight, 12-20% by dry weight, 15-25% by dry weight, 20-45% by dry weight, 30-50% by dry weight, 40-65% by dry weight, 60-85% by dry weight, 62-75% by dry weight, 70-85% by dry weight, or 80-90% by dry weight. In some embodiments, the composition comprises magnesium glycerophosphate at an amount of at least 5% by dry weight, at least 10% by dry weight, at least 15% by dry weight, at least 20% by dry weight, at least 25% by dry weight, at least 35% by dry weight, at least 45% by dry weight, at least 55% by dry weight, at least 65% by dry weight, at least 75% by dry weight, at least 85% by dry weight, at least 89% by dry weight, or any range or value therebetween. In some embodiments, the composition comprises magnesium glycerophosphate at an amount of 90% by dry weight at most.

Coatings

In some embodiments, the composition further comprises a coat or shell. As used herein the term “coat” or “shell” encompasses any material (e.g., a barrier material or a glazing material) that is used to cover, for example, a nutraceutical compound or a pharmaceutical drug, for oral administration, so as to prevent or protect it from being degraded, dissoluted or disintegration under gastric conditions or environment.

Coating materials are common in the art of pharmaceutical and nutraceutical oral administration and would be apparent to a skilled artisan. Non-limiting examples of coating or shelling materials include, but are not limited to, lecithin, methyl acrylate-methacrylic acid copolymers, cellulose acetate phthalate (CAP), cellulose acetate succinate, hydroxypropyl methyl cellulose (HPMC), HPC, hydroxypropyl methyl cellulose acetate succinate (hypromellose acetate succinate), polyvinyl acetate phthalate (PVAP), methyl methacrylate-methacrylic acid copolymers, shellac, cellulose acetate trimellitate, sodium alginate, zein, ethylcellulose, medium chain triglycerides (e.g. such as in coconut), oleic acid, sodium alginate, stearic acid, glycerol, 1,2,3-triacetoxypropane (i.e., triacetin or glycerin triacetate), titanium dioxide, and the like.

In some embodiments, the composition comprises a coat material in an amount of 0.5-1.5% by weight, 1-2% by weight, 1-3% by weight, 1-4% by weight, 1-5% by weight, 2-3% by weight, 2-4% by weight, 2-5% by weight, 3-4% by weight, 3-5% by weight, or 4-8% by weight. Each possibility represents a separate embodiment of the invention. In some embodiments, the composition comprises a coat material in an amount of at least 1% by weight, at least 1.5% by weight, at least 2% by weight, at least 2.5% by weight, at least 3% by weight, at least 3.5% by weight, at least 4% by weight, at least 4.5% by weight, at least 5% by weight, or at least 8% by weight, and any value or range therebetween.

Additive

In some embodiment, the composition further comprises one or more additives. In some embodiments, the additive is selected from: amino acid, acidulent, acidity regulator, bulking agent, coloring agent, mineral, vitamin, emulsifier, flavoring agent, flavor enhancing agent, glazing agent, preservative, stabilizer, gelling agent, sweetening agent, thickening agent, and any combination thereof. These additives may be included in the core or coat. The term “core” as used herein, refers to any part of the composition excluding the coat.

In some embodiments, the coloring agent is an anthocyanin or a combination thereof. in some embodiments, the coloring agent is present in the coat of the composition, in the core of the composition, or both. In some embodiments, the composition, e.g., the core of the composition, comprises a coloring agent in the amount ranging from 0.5-5%, by weight, 0.75-3.5%, by weight, 1.5-4.5%, by weight, 0.3-1.5%, by weight. Each possibility represents a separate embodiment of the invention. In some embodiments, the coat of the composition comprises a coloring agent in an amount ranging from 0.01-1%, by weight.

As used herein, an amino acid is a single amino acid. In some embodiments, the amino acid is not bound by a peptide bond. In some embodiments, the amino acid is not a part of a di-peptide, a peptide, a polypeptide, or a protein. In some embodiments, the amino acid is exogenous to the microalgae. In some embodiments, the amino acid is not derived from the microalgae. In some embodiments, the amino acid is a purified amino acid. In some embodiments, the amino acid is synthetically produced. In some embodiments, the amino acid is produced in vitro. In some embodiments, the amino acid is protein-free grade. In some embodiments, the amino acid is essentially devoid of animal-derived compounds, such as peptides, hormones, lipids, polysaccharides, and the like.

In some embodiments, the composition further comprises one or more amino acids. As used herein, one or more amino acids comprises 2-4, 2-5, or 2-9 amino acids. In some embodiments, the composition further comprises at least 2, at least 4, at least 5, at least 7, at least amino acids, and any value and range therebetween. Each possibility represents a separate embodiment of the invention. In some embodiments, the amino acid is a L-amino acid enantiomer. In some embodiments, the amino acid is a D-amino acid enantiomer. In some embodiments, the composition further comprises D-amino acid enantiomer, L-amino acid enantiomer, or a combination thereof. In some embodiments, the composition further comprises one or more D-amino acid enantiomers, and one or more L-amino acid enantiomers, or any combination thereof. In some embodiments, the composition further comprises L-Leucine. In some embodiments, the composition further comprises L-Valine. In some embodiments, the composition further comprises L-Leucine and L-Valine.

In some embodiments, the composition further comprises an amino acid in an amount of at least 0.5% by weight, at least 1% by weight, at least 1.5% by weight, at least 2% by weight, at least 2.5% by weight, or at least 3% by weight, and any range therebetween. In some embodiments, the composition comprises an amino acid in an amount of 0.5-1.5% by weight, 1-3% by weight, or 1.5-3% by weight. Each possibility represents a separate embodiment of the invention.

In some embodiment, the disclosed composition is formulated for oral administration. For oral applications, the composition may be in the form of tablets, caplets or capsules, which can contain any of the ingredients, or compounds mentioned hereinabove. When the dosage unit form is a capsule, it can contain, in addition to materials of the above type, a liquid carrier, such as fatty oil. In addition, dosage unit forms can contain various other materials which modify the physical form of the dosage unit. A tablet comprising the disclosed composition can further be film coated. In some embodiment, oral application of the composition may be in the form of an edible product, such as a chewable tablet.

In some embodiments, the composition is formulated as a nutraceutical composition, a pharmaceutical composition, a cosmeceutical composition, a dietary supplement, or any combination thereof.

For a non-limiting example, the composition may be incorporated in dry formulations of nutritional supplements and packaged in gel capsules, tablets, sachets and the like. In yet another example, the product may be useful in a liquid form or packaging in soft capsules.

In some embodiments, the disclosed composition has an increased shelf life. As used herein, the term “shelf life” refers to the period of time in which a product or commodity can be stored without becoming inadequate for use, sale, or consumed. In some embodiments, the term shelf life refers to that at least 80%, at least 85%, at least 90%, at least 95%, at least 97%, or at least 99% of the composition is stable in a tablet, and any value and range therebetween. In some embodiments, 80-92%, 85-95%, 93-97%, or 96-100% of the composition is stable in a tablet. Each possibility represents a separate embodiment of the invention. In some embodiment, the term “shelf life” refers to the concentration of the active ingredient. In some embodiments, the active ingredient is astaxanthin. In some embodiments, the shelf life is a measurement of the weight % of the active ingredient.

In some embodiments, the composition has a shelf life of at least 3 months, at least 6 months, at least 8 months, at least 12 months, at least 16 months, at least 20 months, or at least 24 months, and any value or range therebetween. Each possibility represents a separate embodiment of the invention. In some embodiments, the composition has a shelf life of 3-8 months, 5-10 months, 9-14 months, 12-16 months, 15-18 months, 17-22 months, or 20-26 months. Each possibility represents a separate embodiment of the invention. In some embodiments, shelf life duration as disclosed hereinabove is determined in room temperature.

In some embodiments, the composition of the invention comprises high level of stable astaxanthin. The term “stable astaxanthin” encompasses chemically reduced or non-oxidized astaxanthin. The terms “chemically reduced” and “non-oxidized” are used herein interchangeably. In some embodiments, the term “stable” refers to that the amount of astaxanthin formulated into the compositions maintained for a period as disclosed hereinabove. In some embodiments, a maintained amount as disclosed herein is at least 90%, at least 95%, at least 99% of the amount of astaxanthin formulated into the composition, and any value and range therebetween. Each possibility represents a separate embodiment of the invention. In some embodiments, a maintained amount as disclosed herein is 75-85%, 80-92%, 90-95%, 93-98%, or 97-100% of the amount of astaxanthin formulated into the composition. Each possibility represents a separate embodiment of the invention.

In some embodiments, the composition comprises at least 70%, at least 80%, at least 90%, at least 95%, at least 99%, or 100% chemically reduced astaxanthin, and any value and range therebetween. In some embodiments, the composition comprises 65-80%, 75-95%, 90-99%, or 95-100% chemically reduced astaxanthin. Each possibility represents a separate embodiment of the invention.

In some embodiments, the composition comprises stable astaxanthin for a period of at least 2 weeks, at least 3 weeks, at least 4 weeks, at least 2 months, at least 4 months, at least 6 months, at least 12 months, at least 15 months, at least 18 months, at least 24 months, and any value and range therebetween. In some embodiments, the composition comprises stable astaxanthin for a period of 3-5 weeks, 4-6 weeks, 5-8 weeks, 1-3 months, 2-5 months, 4-6 months, 6-9 months, 9-12 months, 10-16 months, 15 to 20 months, 18-24 months, or 22-28 months. Each possibility represents a separate embodiment of the invention. In some embodiments, duration of astaxanthin stability as disclosed hereinabove is determined in room temperature.

As used herein, increased shelf life is relative to control. In some embodiments, a control comprises a composition comprising the compounds as disclosed hereinabove but not in the amounts or ratios disclosed hereinabove. In some embodiments, a control comprises a composition missing at least one of the compounds as disclosed hereinabove. In some embodiments, a control comprises the compounds of the composition of the invention but further includes other agents, materials, or elements. In some embodiments, a control comprises a composition missing at least one of the compounds as disclosed hereinabove, and wherein the compounds are not in the amounts and ratios disclosed hereinabove. In some embodiments, a control comprises the compounds of the composition of the invention but further includes other agents, materials, or elements, and wherein the compounds are not in the amounts and ratios disclosed hereinabove.

Non-limiting examples of suitable pharmaceutical carriers are described in, e.g., Martin, 1990, Remington's Pharmaceutical Sciences, 17th ed. (Mack Pub. Co., Easton, Pa.). Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The compositions of the invention may also contain pH buffering reagents and wetting or emulsifying agents. The compositions may further contain other active compounds providing supplemental, additional, or enhanced therapeutic functions. The compositions may also be included in a container, pack, or dispenser together with instructions for administration.

Non-limiting examples of suitable acceptable salts of the compounds of the invention may also be included in the compositions. Examples of salts include salts of inorganic acids (such as, e.g., hydrochloric, hydrobromic, phosphoric, metaphosphoric, nitric, and sulfuric acids) and of organic acids (such as, e.g., acetic, benzenesulfonic, benzoic, citric, ethanesulfonic, fumaric, gluconic, glycolic, isethionic, lactic, lactobionic, maleic, malic, methanesulfonic, succinic, p-toluenesulfonic, and tartaric acids). Other suitable acceptable basic salts include ammonium salts, alkali metal salts (such as, e.g., sodium and potassium salts) and alkaline earth metal salts (such as, e.g., magnesium and calcium salts). Furthermore, the compounds of the invention may be present as a hydrate or hemihydrate (of the compound or of its salt).

Examples of suitable aqueous and nonaqueous carriers, diluents, solvents or vehicles include water (e.g., pyrogen-free water), ethanol, polyols (such as glycerol, propylene glycol, polyethylene glycol, and the like), and suitable mixtures thereof, vegetable oils (such as olive oil, cotton seed oil, palm oil, etc.), and injectable organic esters such as ethyl oleate. Proper fluidity can be maintained, for example, by the use of coating materials such as lecithin, by the maintenance of the required particle size in the case of dispersions, and by the use of surfactants. These compositions may also contain adjuvants, antibacterial and antifungal agents, flavoring agents, biodegradable polymers, etc.

The compositions of this invention can be provided or administered to mammals (e.g., humans, etc.) in any suitable way including, e.g., orally, parenterally, intracisternally, intraperitoneally, topically, etc.

In certain embodiments the solid composition formulated as tablets contain in addition to the active compound suitable excipients including, but not limited to, starches, gum arabic/gum acacia, calcium silicate, microcrystalline cellulose, polyvinylpyrrolidone, cellulose, water, syrup, and methylcellulose. The formulations can additionally include lubricating agents such as, for example, talc, and mineral oil; wetting agents; emulsifying and suspending agents; preserving agents such as methyl- and propyl hydroxybenzoates; sweetening agents; or flavoring agents. Polyols, buffers, and inert fillers may also be used. Examples of polyols include, but are not limited to: mannitol, sorbitol, xylitol, sucrose, maltose, glucose, lactose, dextrose, and the like. Suitable buffers encompass, but are not limited to, phosphate, citrate, tartrate, succinate, and the like. Other inert fillers, which may be used, encompass those which are known in the art and are useful in the manufacture of various dosage forms. If desired, the solid compositions may include other components such as bulking agents and/or granulating agents, and the like.

In some embodiments, there is provided a method for increasing the stability of astaxanthin in a composition, the method comprising: mixing a microalgae powder comprising astaxanthin, and magnesium powder at a ratio ranging from 10:1 to 1:10, wherein the magnesium is ionically bound to an anion comprising an oxygen ion, thereby increasing the stability of astaxanthin in the composition.

As used herein, the term “increasing stability of astaxanthin” relates to astaxanthin level and encompasses any one of: maintaining the level of astaxanthin, preventing degradation and/or oxidation of astaxanthin, safeguarding of astaxanthin, stabilizing of astaxanthin, and any combination thereof.

In some embodiments, the method comprises mixing the microalgae powder comprising astaxanthin, and magnesium (for example, magnesium glycerophosphate), at a ratio of 10:1, 9:1, 8:1, 7:1, 6:1, 5:1.4:1, 3:1, 2:1, 1:1, 1:2, 1:3, 1:4, 1:5, 1:6, 1:7, 1:8, 1:9, 1:10, or any value or range therebetween. In some embodiments, the method comprises mixing the microalgae powder comprising astaxanthin, and magnesium (for example, magnesium glycerophosphate), at a ratio ranging from 10:1 to 1:10, 9:1 to 1:9, 8:1 to 1:8, 7:1 to 1:7, 6:1 to 1:6, 5:1 to 1:5, 4:1 to 1:4, 3:1 to 1:3, 2:1 to 1:2, 10:1 to 1:10, 10:1 to 1:10, 10:1 to 1:10, 10:1 to 1:10, 10:1 to 1:10, 10:1 to 1:10. Each possibility represents a separate embodiment of the invention.

In some embodiments, the method comprises increasing the shelf-life of astaxanthin. In some embodiments, performing the method of the invention results in astaxanthin with increased shelf-life. In some embodiments, astaxanthin increased shelf-life is relative to astaxanthin shelf-life in a composition other than the composition disclosed herein. In some embodiments, astaxanthin shelf life is increased in the composition of the invention compared to astaxanthin shelf-life in a composition other than the composition of the invention. In some embodiments, the composition of the invention does not reduce the shelf-life of astaxanthin. In some embodiments, the method comprises reducing the rate of astaxanthin oxidation. In some embodiments, the method comprises reducing the amount of oxidized astaxanthin. In some embodiments, the method comprises increasing the amount of chemically reduced astaxanthin. In some embodiments, the method comprises increasing the amount of stable astaxanthin. In some embodiments, the method comprises increasing the shelf life of a composition comprising microalgae powder comprising astaxanthin, and magnesium. In some embodiments, the term “increasing” used herein, is relative to control.

As used herein, the term “about” when combined with a value refers to plus and minus 10% of the reference value. For example, a length of about 1,000 nanometers (nm) refers to a length of 1,000±100 nm.

It is noted that as used herein and in the appended claims, the singular forms “a”, “an”, and “the” include plural referents unless the context clearly dictates otherwise. Thus, for example, reference to “a compound” includes a plurality of such compounds and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

### EXAMPLES

Materials and Methods

Determination of Natural Astaxanthin Complex in H. pluvialis Whole Algae Powder

Concentration of natural astaxanthin complex was measured in natural astaxanthin powder.

Equipment and Parameters

Centrifuge, Spectrophotometer (at 472 nm), 15 ml glass test tubes+screw cups (suitable for centrifugation), mortar and pestle, vortex mixer, glass pipette, transfer pipette, analytical scales, and Acetone (CP grade).

Procedure

5-10 mg of the powder were transferred to each 15 ml glass centrifuge tube (three independent replicates are recommended). Ten 10 ml of acetone were added, the tubes were capped, vortexed thoroughly (30 seconds), and left at room temperature for 5 minutes while being protected from light. Vortex and room temperature incubation was repeated 3 times. Thereafter, tubes were centrifuged (5 min at 3,000 rpm). Absorbance was measured using a spectrophotometer at a wavelength of 472 nm (using acetone as ‘blank’). Optical density (OD) readings were kept in the linear value (0.2-1.5).

Concentrations were calculated (and recorded), as follows:

Astaxanthin Complex (μg/ml)=OD×KACETONE

Astaxanthin Complex (%)=[OD×KACETONE×F]/[1,000×W]×100 (for %)

Astaxanthin (%)=Astaxanthin Complex (%)×Astaxanthin purity (Measured by HPLC)

KACETONE is the coefficient for astaxanthin in acetone, as determined by standard calibration curve. The coefficient KACETONE (4.7) is the inverse average slope of calibration curves, performed using astaxanthin standard concentrations of 1-15 μg/ml. Optimal (linear) O.D. range is 0.2-1.5.

W—sample weight (mg); F—dilution factor, or the volume of the sample (10 ml). Result reflects the average of 2 independent replicates.

### Example 1

To formulate a successful whole algae tablet, the powder mixture should be composed of a mechanically stable mixture to withstand the process of tabletation (e.g., pressing a composition to obtain a tablet), among which compression forces are applied which can lead to tablet disintegration. The manufactured tablets should also retain the activity of the active ingredient(s) entrapped within and enable resistance to oxidation so as to provide extended shelf life. The formulated tablet can further comprise a coat or a shell.

The biotechnological challenge of formulating the tablet composition was to find the right ingredient and excipient combination which provides initial mechanical stability followed by fine tuning of the composition so as to provide the highest astaxanthin stability over time under either real time or accelerated stability studies.

As expressed in the tables herein below, “%” refers to % weight.

TABLE 1
Formulations 1-3 consisted of:

| | Formulation 1 | Formulation 2 | Formulation 3 |
|---|---|---|---|
| Haematococcus Powder | 26.66% | 26.70% | 28.90% |
| (Active Ingredient) | | | |
| Silicon dioxide | 1.00% | 1.00% | 1.00% |
| Stearic Acid | 1.00% | 1.00% | 1.00% |
| PVP | 6.00% | | |
| HPC | | 4.00% | 4.00% |
| Sodium croscarmellose | 3.00% | 3.00% | 3.00% |
| Magnesium Stearate | 1.00% | 1.00% | 1.00% |
| Rosemary oil powder | | | 0.30% |
| DCP | 61.34% | 63.30% | 60.80% |
| Total | 100.00% | 100.00% | 100.00% |

TABLE 2
Formulations 4-6 consisted of:

| | Formulation 4 | Formulation 5 | Formulation 6 |
|---|---|---|---|
| | Trial 4 | Trial 5 | Trial 6 |
| Haematococcus Powder | 19% | 19% | 19% |
| (Active Ingredient) | | | |
| Silicon dioxide | 1% | 1% | 1% |
| Maltodextrin | 20% | 10% | N/A |
| Gum acacia | 20% | 10% | N/A |
| Inulin | 40% | 60% | 80% |
| Total | 100% | 100% | 100% |

TABLE 3
Formulations I-VIII consisted of:

| | Formula- | Formula- | Formula- | Formula- | Formula- | Formula- | Formula- | Formula- |
|---|---|---|---|---|---|---|---|---|
| | tion I | tion II | tion III | tion IV | tion V | tion VI | tion VII | tion VIII |
| | Trial 1 | Trial 2 | Trial 3 | Trial 4 | Trial 5 | Trial 6 | Trial 7 | Trial 8 |
| Haematococcus | 29.1 | 28.9 | 28.9 | 29.2 | 29.2 | 29.2 | 28.9 | 28.9 |
| Powder (Active | | | | | | | | |
| Ingredient) (gr) | | | | | | | | |
| Stearic acid | 3.1 | 2.99 | 2.99 | 3.02 | 11.07 | 11.07 | 5.98 | 5.98 |
| (gr) | | | | | | | | |
| HPC (gr) | 4.15 | 3.98 | 3.98 | 4.02 | 4.02 | 4.02 | 3.98 | 3.98 |
| Sodium | 3.15 | 2.99 | 2.99 | 3.02 | 3.02 | 3.02 | 2.99 | 2.99 |
| croscarmellose | | | | | | | | |
| Rosemary oil | 0.35 | 0.29 | 0.299 | 0.3 | 0.3 | 0.3 | 0.29 | 0.29 |
| powder (gr) | | | | | | | | |
| Silicon dioxide | 0.15 | 2.99 | 2.99 | 2.01 | 2.01 | 2.01 | 1.99 | 199 |
| (gr) | | | | | | | | |
| MCC (gr) | 60 | 57.8 | 51.8 | | | | | |
| TCP (gr) | | | 5.98 | 4.02 | 6.04 | 6.04 | | 1.99 |
| Calcium | | | | 54.38 | 44.3 | 44.3 | | 7.97 |
| Carbonate (gr) | | | | | | | | |
| Inulin (gr) | | | | | | | 19.9 | 9.97 |
| Gum acacia | | | | | | | 35.8 | 35.8 |
| (gr) | | | | | | | | |
| Results | No flow | No flow | Flow, weight | Mechanically | Medium | Reground, second | Tablet | Tablet |
| | | | too low (72%) | unstable powder | mechanical | press, table | mechanically | mechanically |
| | | | | | stability, | mechanically | unstable, weight | unstable, weight |
| | | | | | weight too low | unstable | too low (74%) | too low |

TABLE 4
Formulations IX-XIII consisted of:

| | Formulation IX | Formulation X | Formulation XI | Formulation XII | Formulation XIII |
|---|---|---|---|---|---|
| | Trial 1 | Trial 1 | Trial 2 | Trial 3 | Trial 4 |
| Haematococcus Powder | 145 | 142.5 | 142.5 | 135 | 135 |
| (Active Ingredient) (gr) | | | | | |
| Stearic acid (gr) | 30 | | | | |
| HPC (gr) | 20 | | | | |
| Sodium croscarmellose | 15 | | | | |
| Rosemary oil | 1.5 | 2.25 | 2.25 | 2.25 | 2.25 |
| powder (gr) | | | | | |
| Silicon dioxide (gr) | 10 | 7.5 | 7.5 | 7.5 | 7.5 |
| TCP (gr) | 10 | | | | |
| Calcium Carbonate (gr) | 40 | | | | |
| Inulin (gr) | 50 | 597.75 | 597.75 | 605 | 605 |
| Gum acacia (gr) | 180 | | | | |
| Results | Formulation VIII | Crumbles, | Tablet mechanically | Crumbles | Tablet mechanically |
| | trial 8, re-ground, | mechanically | unstable, no | mechanically, | unstable, no |
| | second press, | unstable | flow, weight | unstable | flow |
| | tablet mechanically | | too low (54%) | | |
| | unstable | | | | |

TABLE 5
Formulations XIV-XVI and 7 consisted of:

| | Formulation XIV | | Formulation XV | | Formulation XVI | | Formulation 7 | |
|---|---|---|---|---|---|---|---|---|
| | % | mg/Tab | % | mg/Tab | % | mg/Tab | % | mg/Tab |
| Haematococcus Powder | 19 | 142.5 | 19 | 142.5 | 19 | 142.5 | 21 | 142.5 |
| (Active Ingredient) | | | | | | | | |
| Maltodextrin | | | | | | | 20 | 136 |
| Gum acacia | | | | | | 20 | 136 | |
| Carnauba wax | | | 1 | 7.5 | 3 | 22.5 | 1 | 6.8 |
| Silicon dioxide | 1 | 7.5 | 1 | 7.5 | 1 | 7.5 | 1 | 6.8 |
| Inulin | 80 | 600 | 79 | 592.5 | 77 | 577.5 | 37 | 251.9 |
| Total | 100 | 750 | 100 | 750 | 100 | 750 | 100 | 680 |
| Results | Slightly | | Slightly | | Pass, | | Slightly | |
| | soft, | | soft, | | 700 mg | | soft, | |
| | 680 mg | | 680 mg | | | | 640 mg | |

TABLE 6
Formulation 8 consisted of:

| | % | mg/Tab |
|---|---|---|
| Haematococcus Powder | 22 | 142 |
| (Active Ingredient) | | |
| Maltodextrin | 20 | 129 |
| Gum acacia | 20 | 129 |
| Carnauba wax | | |
| Silicon dioxide | 1 | 6.45 |
| Inulin | 37 | 238.55 |
| Total | 100 | 645 |
| Results | Slightly soft, | |
| | 630 mg | |

TABLE 7
Formulation 9 consisted of:

| | % | mg/Tab |
|---|---|---|
| Haematococcus Powder | 18.9 | 132.3 |
| (Active Ingredient) | | |
| Maltodextrin | 0 | 0 |
| Gum acacia | 0 | 0 |
| Carnauba wax | 2.99 | 20.9 |
| Silicon dioxide | 1.49 | 10.5 |
| Inulin | 76.6 | 536.3 |
| Total | 100 | 700 |

Formulations I to XVI failed to meet the requirements of structural integrity needed for the tableting process. These formulations were characterized as having: no flow in the processing conveyer, low mechanical stability, low tablet weight, or a combination thereof.

Contrary to the above, formulations number 1-9 were found satisfactory with respect to mechanical stability, and processability (e.g., flow).

### Example 2

After formulations 1-9 were found to meet the physical requirements of a tablet, the stability of astaxanthin over time was examined under vacuum conditions. The results are presented in the tables hereinbelow.

TABLE 8
Astaxanthin stability in formulation 1:
No coating, formulation 1

| Number of days | % Astaxanthin remaining |
|---|---|
| 0 | 100 |
| 15 | 83.7 |
| 31 | 70.6 |

TABLE 9
Astaxanthin stability in formulation 2:
Coated, formulation 2

| Number of days | % Astaxanthin remaining |
|---|---|
| 0 | 100 |
| 30 | 83.9 |
| 55 | 69.8 |

TABLE 10
Astaxanthin stability in formulation 3:
Formulation 3

| Number of days | % Astaxanthin remaining |
|---|---|
| 0 | 100 |
| 28 | 77.6 |

TABLE 11
Astaxanthin stability in formulation 4:
No coating, vacuum, formulation 4

| Number of days | % Astaxanthin remaining |
|---|---|
| 0 | 100 |
| 7 | 100 |
| 14 | 100 |
| 21 | 99.9 |
| 35 | 100 |

TABLE 12
Astaxanthin stability in formulation 5:
No coating, vacuum, formulation 5

| Number of days | % Astaxanthin remaining |
|---|---|
| 0 | 100 |
| 7 | 98.4 |
| 14 | 95.5 |
| 21 | 97.8 |
| 35 | 94.3 |

TABLE 13
Astaxanthin stability in formulation 6:
No coating, vacuum, formulation 6

| Number of days | % Astaxanthin remaining |
|---|---|
| 0 | 10 |
| 7 | 95.9 |
| 14 | 92.4 |
| 21 | 96.4 |
| 35 | 96.8 |

TABLE 14
Astaxanthin stability in formulation 7, under vacuum conditions:
No coating, vacuum, formulation 7

| Number of days | % Astaxanthin remaining |
|---|---|
| 0 | 100 |
| 10 | 93.1 |
| 14 | 100 |
| 23 | 96.2 |
| 30 | 100 |

TABLE 15
Astaxanthin stability in formulation 7, under aerated conditions:
No coating, stored in a bottle, formulation 7

| Number of days | % Astaxanthin remaining |
|---|---|
| 0 | 100 |
| 14 | 99.2 |
| 31 | 100.8 |

TABLE 16
Astaxanthin stability in formulation 8
(2 duplicates), under vacuum conditions:
No coating, vacuum, formulation 8

| Number of days | % Astaxanthin remaining |
|---|---|
| 0 | 100 |
| 31 | 97.8 |
| 62 | 99.3 |
| 90 | 100 |

TABLE 17
Astaxanthin stability in formulation 8, under aerate conditions:
No coating, stored in a bottle, formulation 8

| Number of days | % Astaxanthin remaining |
|---|---|
| 0 | 100 |
| 31 | 100 |
| 62 | 97.8 |
| 90 | 97.1 |

TABLE 18
Astaxanthin stability in formulation 9, under vacuum conditions:
No coating, vacuum, formulation 9

| Number of days | % Astaxanthin remaining |
|---|---|
| 0 | 100 |
| 31 | 90.6 |
| 62 | 92.8 |
| 90 | 94.2 |

TABLE 19
Astaxanthin stability in formulation 9, under aerated conditions:
No coating, stored in a bottle, formulation 9

| Number of days | % Astaxanthin remaining |
|---|---|
| 0 | 100 |
| 7 | 91.3 |
| 13 | 84.3 |

Astaxanthin stability was found be substantially lower in formulations 1-3, showing a decrease of 20-30% compared to the amount initially formulated into the tablet. Contrary to the above, formulations 4-9 showed high efficiency in maintaining high levels of astaxanthin, which were generally unaffected throughout the experimentation periods.

Astaxanthin was found to be highly stable in formulations 7-9, both under vacuum and aerated conditions. Astaxanthin was found to be highly stable (˜100%) in formulation 7 for a period of at least 30 days, regardless if the tablet was under vacuum or aerated conditions. In formulation 8, astaxanthin was found to be highly stable for a period of at least 90 days, showing 100% stability when tablet was under vacuum conditions or over 90% stability when tablet was under aerated conditions. In formulation 9, astaxanthin was found to be highly stable (>90%) when the tablet was under vacuum conditions for a period of at least 90 days. Therefore, tables comprising the disclosed formulations, such as 7-9, are determined as a highly-stable astaxanthin-source with long shelf life (Tables 14-19).

### Example 3

### Stability of Astaxanthin in Combination with Magnesium Stearate

The stability of astaxanthin (from Haematococcus pluvialis biomass powder) (“biomass”) was evaluated by mixing 80 gr of biomass with 20 gr of magnesium stearate followed by incubation at 60° C. under vacuum conditions and darkness. Samples were removed from incubation on days 0, 7 and 14 days, dissolved with 100% acetone and the % of chemically-reduced (e.g., non-oxidized) astaxanthin was determined by measuring the absorbance at a wavelength of 472 using ultraviolet-visible spectrophotometry (UV-VIS) nm (as described). The experiment was performed using two biomass batches (e.g., AST1-35-3, and AST1-36-3), and the results are summarized hereinbelow (Table 20).

TABLE 20
Stability of astaxanthin in the presence of magnesium stearate

| | |
|---|---|
| ASTI-35-3 | ASTI-36-3 |

| | |
|---|---|
| Number of days | % Astaxanthin remaining |

| | | |
|---|---|---|
| 0 | 100 | 100 |
| 7 | 82.1 | 72.4 |
| 14 | 79.7 | 78.3 |

The astaxanthin stability deteriorated well below 90% within less than a week, therefore this formulation was concluded by the inventors as not adequate.

### Example 4

### Stability of Astaxanthin in Combination with Magnesium Citrate

The stability of astaxanthin was further evaluated by mixing 6 gr of biomass with 94 gr of magnesium citrate and incubating the mixture at 40° C. under vacuum conditions and darkness. Samples were removed from incubation on days 0 and 21, dissolved with 100% acetone and the astaxanthin content was determined by measuring the absorbance at a wavelength of 472 nm using UV-VIS (as described). The experiment was performed using two biomass batches (e.g., AST-1-37-4 and AST-1-37-10), and the results are summarized hereinbelow (Table 21).

TABLE 21
Stability of astaxanthin in the presence of magnesium citrate

| | |
|---|---|
| AST-1-37-4 | AST-1-37-10 |

| | |
|---|---|
| Number of days | % Astaxanthin remaining |

| | | |
|---|---|---|
| 0 | 100 | 100 |
| 21 | 66.7 | 82.7 |

The astaxanthin stability deteriorated well below 90% within less than 3 weeks, therefore this formulation was concluded by the inventors as not adequate.

### Example 5

### Stability of Astaxanthin in Combination with Magnesium Oxide

The stability of astaxanthin was evaluated by incubating 300 gr of biomass at 40° C. under vacuum conditions and darkness, either alone (“control) or pre-mixed with 40 gr of magnesium oxide (“experimental group”). Samples were removed from both groups on days 0, 12 and 30, dissolved with 100% acetone and the astaxanthin content was determined by measuring the absorbance at a wavelength of 472 nm using UV-VIS (as described). The results are summarized hereinbelow (Table 22).

TABLE 22
Stability of astaxanthin in the presence of magnesium oxide

| | |
|---|---|
| Experimental group | Control |

| | |
|---|---|
| Number of days | % Astaxanthin remaining |

| | | |
|---|---|---|
| 0 | 100 | 100 |
| 12 | 67 | 97.5 |
| 30 | 32.9 | 98.2 |

While astaxanthin stability remained above 90% in the control, it deteriorated well below 90% in the presence of magnesium oxide within less than 12 days, therefore this formulation was concluded by the inventors as not adequate.

### Example 6

### Stability of Astaxanthin in Combination with Magnesium Glycerophosphate

The stability of astaxanthin was further evaluated by mixing 25 gr of biomass with 75 gr of magnesium glycerophosphate and incubating the mix at 40° C. under vacuum conditions and darkness. Samples were removed from incubation on days 0, 13, 31, 40 and 61, dissolved with 100% acetone and the astaxanthin content was determined by measuring the absorbance at a wavelength of 472 nm using UV-VIS (as described).

TABLE 23
Stability of astaxanthin in the presence
of magnesium glycerophosphate

| | Biomass + Magnesium glycerophosphate mixture |
|---|---|
| Number of days | % Astaxanthin remaining |
| 0 | 100 |
| 13 | 93.3 |
| 31 | 94.5 |
| 40 | 94.5 |
| 61 | 95.8 |

The astaxanthin stability remained well above 90% for a period of over 2 months, therefore this formulation was concluded by the inventors as adequate and stable.

### EXAMPLE 7

### Comparative Analysis of Astaxanthin Stability in the Presence of Different Magnesium Salts

The stability of astaxanthin was compared between two types of magnesium salts by mixing 25 gr of biomass with 75 gr of either magnesium glycerophosphate or magnesium oxide and incubating the mixtures at 40° C. under vacuum conditions and darkness. Samples were removed from both mixtures on days 0, 14, 31 and 45, dissolved with 100% acetone and the astaxanthin content was determined by measuring the absorbance at a wavelength of 472 nm using UV-VIS (as described). The results are summarized hereinbelow (Table 24).

TABLE 24
Stability of astaxanthin in the presence of
magnesium oxide or magnesium glycerophosphate

| | |
|---|---|
| | Magnesium |
| Magnesium oxide | glycerophosphate |

| | |
|---|---|
| Number of days | % Astaxanthin remaining |

| | | |
|---|---|---|
| 0 | 100 | 100 |
| 14 | 97.4 | 100 |
| 31 | 83.8 | 100 |
| 45 | 55.9 | 100 |

In the biomass-magnesium glycerophosphate mixture, astaxanthin levels were unaffected throughout the experiment (45 days), therefore this formulation was concluded by the inventors as adequate and stable. On the contrary, the magnesium oxide mixture which not only found to be non-homogenous, but the astaxanthin level deteriorated well below 90% within less than a month, therefore this formulation was concluded by the inventors as not adequate.

### EXAMPLE 8

### Stability of Astaxanthin in Combination with Magnesium

The stability of astaxanthin was evaluated by mixing 16 gr of 3% Astaxanthin powder with 80 gr of magnesium pidolate and incubating the mix in closed aluminum bags. Samples were removed from incubation on days 0, 35, 42, 55, 57, 90, 118 and 124, and the chemically-reduced (non-oxidized) astaxanthin % was determined as described above.

TABLE 25
Stability of astaxanthin in the presence of magnesium pidolate

| | Astaxanthin + Mg Pidolate | Astaxanthin alone |
|---|---|---|
| Time point (Mo.) | % Astaxanthin remaining | |
| 0 | 100% | 100% |
| 1.17 | | 92.2% |
| 1.40 | 93.4% | |
| 1.83 | | 94.5% |
| 1.90 | 88.5% | |
| 3.93 | | 86.4% |
| 4.13 | 85.2% | |

The astaxanthin stability remained well above 85% for a period of over 4 months in the presence of magnesium pidolate, therefore this formulation was concluded by the inventors as adequate and stable.

### EXAMPLE 9

Assessment of Exercise Performance and Exercise Related General and Muscle Fatigue/Pain

The objective was to compare the effect of a “Max” composition on muscle pain, cramps, recovery and general feeling of subjects (active women and men; age 35-65) who exercise regularly, compared to “placebo”.

TABLE 26
“Max”

| Active ingredient tablets | Amount (mg) | Percentage |
|---|---|---|
| 3% Astaxanthin powder | 98 | 8.69 |
| Silica dioxide | 18 | 1.60 |
| Inulin | 422 | 37.41 |
| MG glycerophosphate | 500 | 44.33 |
| B6 | 2 | 0.18 |
| HPC | 88 | 7.80 |
| Total | 1,128 | 100 |

*White coating - Seppic LP770 3-5% gain weight

TABLE 27
Placebo

| | Amount (mg) | Percentage |
|---|---|---|
| Calcium carbonate | 1046 | 77.4 |
| Stearic acid | 203 | 15.0 |
| MCC | 68 | 5.0 |
| Magnesium stearate | 20 | 1.5 |
| Silica dioxide | 14 | 1.0 |
| Total | 1,351 | 100 |

*Un-coated

Eighty (80) subjects were recruited into each experimental arm (placebo or “Max” intake). Self-intake for 8 weeks. Subjects were asked to complete an online survey to monitor their experience, level of pain, cramps, recovery and general feeling.

Outcome Measures—assessment of pain after and or during exercise (at least 2 questionnaires completed per week).

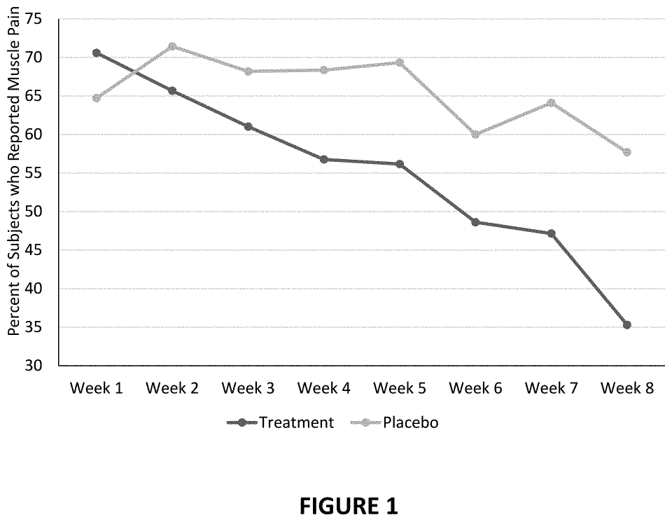

The percent of subjects that have reported pain per treatment week (1-8) was calculated for the two treatment groups. While the Max treatment group showed a substantial reduction in the number of subjects reporting pain, the placebo group showed only a mild reduction (FIG. 1). The difference between the two treatment groups was statistically significant at weeks 7 (p=0.0469) and 8 (p=0.0081).

### Example 10

### Coating Improves Tablet Stability and Appearance

A single batch of tablets was coated in 2 ways: (1) 0.5% gain weight of aqua solution of HPC; (2) 0.8% gain weight of aqua solution of HPC+anthocyanins color (HPC+color) Both batched were coated using the same coating parameters. The coated tablets were stored in sealed bottles and tested for % active ingredient remaining over time. The results are summarized in Table 28.

TABLE 28
Results summary:

| Month | Tablet + HPC + color | Tablet + HPC |
|---|---|---|
| 0 | 100% | 100% |
| 1 | 96% | 100% |
| 2 | 99% | 100% |
| 3 | 94% | 99% |
| 4 | 92% | 0%* |
| 5 | 94% | N/A |
| 6 | 87% | N/A |

*White spots

After 4 months tablets coated with aqua solution of HPC (without anthocyanins color) presented with white spots which disqualified them. These results show that the anthocyanins color prevents oxidation of the active ingredient.

While certain features of the invention have been described herein, many modifications, substitutions, changes, and equivalents will now occur to those of ordinary skill in the art. It is, therefore, to be understood that the appended claims are intended to cover all such modifications and changes as fall within the true spirit of the invention.

1.-40. (canceled)

41. A composition comprising:
a) a microalgae powder in an amount of 5-30% by weight; and
b) magnesium.

42. The composition of claim 41, further comprising a filler, wherein the combination of said magnesium and said filler are in the amount of 60-85, by weight.

43. The composition of claim 41, being in the form of a tablet, and optionally being formulated for oral administration.

44. The composition of claim 41, having increased stability for a period of at least 6 months.

45. The composition of claim 41, further comprising: an excipient, a wax material, an emulsifier, an antioxidant, or a combination thereof.

46. The composition of claim 41, wherein said microalgae powder comprises at least one carotenoid.

47. The composition of claim 46, wherein said at least one carotenoid is selected from the group consisting of: astaxanthin, lutein, and fucoxanthin.

48. The composition of claim 47, wherein said astaxanthin is present in said composition in an amount of 1-8%, by weight.

49. The composition of claim 42, wherein said filler is selected from the group consisting of: inulin, maltodextrin, gum acacia, and any combination thereof, or optionally wherein said filler comprises a dietary fiber.

50. The composition of claim 49, wherein said inulin is present in said composition in an amount of 35-75%, by weight.

51. The composition of claim 45, wherein said wax material is selected from the group consisting of: carnauba wax, beeswax, candelilla wax, rice wax, lanolin wax, and any combination thereof.

52. The composition of claim 51, wherein said wax material is carnauba wax, and being in said composition in an amount of 1-10%, by weight.

53. The composition of claim 45, wherein any one of:
a) said emulsifier is hydroxypropyl cellulose (HPC), sodium croscarmellose, rice extract, or any combination thereof, and optionally wherein said emulsifier is present in said composition in an amount of 1-12%, by weight;
b) said antioxidant is selected from the group consisting of: sodium ascorbate, carnosic acid, rosemary extract, tocopherol, and any combination thereof, and optionally said antioxidant is present in said composition in an amount of 0.01-2%, by weight;
c) said excipient is silicon dioxide, starch, or a combination thereof, and optionally wherein said excipient is present in said composition in an amount of the amount 1-12%, by weight.

54. The composition of claim 49, wherein said dietary fiber is present in said composition in a ratio ranging from 80:1 (w/w) to 2:1 (w/w) with a wax material.

55. The composition of claim 41, wherein said microalgae powder is present in said composition in an amount of 8-24%, by weight.

56. The composition of claim 41, wherein said magnesium is ionically bound to an oxyanion, optionally wherein said magnesium is magnesium glycerophosphate, and optionally wherein said magnesium is present in said composition in an amount of 1-50%, by weight.

57. The composition of claim 41, wherein said microalgae is Haematococcus pluvialis.

58. The composition of claim 41, further comprising any one of:
a) an amino acid in an amount of 0.5-2%, by weight, optionally wherein said amino acid is selected from the group consisting of: Leucine and Valine;
b) a coat or a shell, wherein said coat or shell is present in said composition in an amount of 0.1-8%, by dry weight;
c) one or more additives, optionally wherein said one or more additives are selected from the group consisting of: a coloring agent, a flavoring agent, a vitamin, and a mineral, optionally wherein said coloring agent is present in said composition in an amount of 0.01-5%, by weight, optionally wherein said coloring agent comprises an anthocyanin, and optionally wherein said flavoring agent is present in said composition in an amount of 0.5-2.5%, by weight.

59. A method for ameliorating or reducing any one of muscle pain and muscle cramps, in a subject in need thereof, the method comprising administering to said subject a therapeutically effective amount of the composition comprising: (a) a microalgae powder in an amount of 5-30% by weight; and (b) magnesium, thereby ameliorating or reducing any one of muscle pain and muscle cramps, in the subject.

60. A process for preparing a composition, the process comprising mixing:
a) a microalgae powder in an amount of 5-30%, by weight; and
b) a filler or
c) a filler and magnesium in an amount of 60-85%, by weight, thereby preparing the composition.
